(19) United States
(12) Patent Application Publication
(10) Pub. No.: US 2024/0273262 A1
(43) Pub. Date: Aug. 15, 2024

(54) METHOD FOR MONITORING BIOMANUFACTURING PROCESS USING METABOLIC NETWORK OF CELLS

(71) Applicant: JIANGNAN UNIVERSITY, Wuxi (CN)

(72) Inventors: Fei LIU, Wuxi (CN); Quan LI, Wuxi (CN); Zhonggai ZHAO, Wuxi (CN); Gongxin LI, Wuxi (CN); Xiaoli LUAN, Wuxi (CN); Zhiguo WANG, Wuxi (CN)

(21) Appl. No.: 18/396,157

(22) Filed: Dec. 26, 2023

Related U.S. Application Data
(63) Continuation of application No. PCT/CN22/73490, filed on Jan. 24, 2022.

(30) Foreign Application Priority Data
Aug. 18, 2021 (CN) 202110950873.9

(51) Int. Cl. G06F 30/27; G16B 5/30; G16B 40/20

(52) U.S. Cl. CPC G06F 30/27; G16B 5/30; G16B 40/20

(57) ABSTRACT

The invention relates to a method for monitoring a biomanufacturing process using a metabolic network of cells, including performing a flux balance analysis based on a metabolic network to obtain a metabolic flux; combining the metabolic flux and a production process variable to form a training dataset, and establishing a biological fermentation process monitoring model and a statistical quantity control limit; and performing online running status monitoring based on the monitoring model by using a production process variable that is acquired in real time and a calculated value of the metabolic flux. For the method for monitoring a biomanufacturing process using a metabolic network of cells of the invention, the method uses detectable or observable information in a microbe manufacturing process, and fully exploits undetectable intracellular metabolic information in a growth and reproduction process of cells, so that running status evaluation and fault monitoring can be implemented more effectively.

This application is a Continuation Application of PCT/CN2022/073490, filed on Jan. 24, 2022, which claims priority to Chinese Patent Application No. 202110950873.9, filed on Aug. 18, 2021, which is incorporated by reference for all purposes as if fully set forth herein.

### FIELD OF THE INVENTION

The present invention relates to the field of industrial biological process control technologies, and in particular, to a method for monitoring a biomanufacturing process using a metabolic network of cells.

### DESCRIPTION OF THE RELATED ART

With the global economic development and social advancement, to meet people's living requirements that grow on a daily basis, a small-batch, high-profit, and multi-strain production manner represented by industrial microbial technologies is attracting considerable attention. A biomanufacturing process is generally performed in a biological reactor, and may obtain products with high economic benefits within a specific period of time in a batch production manner. For example, for the production of a common antibiotic penicillin, exogenously secreted proteins and the like of penicillin are generally obtained through biological fermentation reactions. However, various runaways in production statuses or running faults often occur in a fermentation production process. Without early monitoring, pre-warning, and timely adjustment, the production quality and the yield of a target product may be eventually affected. Therefore, running status evaluation and fault monitoring of a biomanufacturing process are of significant actual engineering value.

Both physicochemical and biochemical reactions occur in a biological fermentation process, and the mechanisms are complex inside cells. During actual production, only basic production process variables such as a temperature, a pH, a ventilation rate, and a stirring power and concentrations of some observable extracellular substance can generally be detected. Microscopic metabolic activities inside cells cannot be observed. As a result, in existing fermentation production running status evaluation technologies, comprehensive analysis is mainly performed according to various process variables and concentrations of substances in a production process, the efficacy and timeliness of monitoring of the technologies need to be improved.

### SUMMARY OF THE INVENTION

For this, a technical problem to be resolved by the present invention is to overcome the problem in the prior art that microscopic metabolic activities inside cells cannot be observed, resulting in inadequacy in existing fermentation production running status evaluation, and provides a method for monitoring a biomanufacturing process using a metabolic network of cells of the present invention. The method uses detectable or observable information in a microbial manufacturing process, and fully exploits undetectable intracellular metabolic information in a growth and reproduction process of cells, so that running status evaluation and fault monitoring of a production and manufacturing process can be implemented more effectively.

To resolve the foregoing technical problems, the present invention provides a method for monitoring a biomanufacturing process using a metabolic network of cells, including the following steps: performing a flux balance analysis based on a metabolic network, to obtain a metabolic flux that reflects a growth and reproduction process of cells; combining the metabolic flux in the cells and a production process variable to form a training dataset, and establishing a biological fermentation process monitoring model and a statistical quantity control limit; and performing online running status monitoring based on the monitoring model and by using a production process variable that is acquired in real time and a calculated value of the metabolic flux.

In an embodiment, a process of the flux balance analysis includes: establishing a flux balance equation; establishing a dynamic relationship between an extracellular metabolite concentration and the metabolic flux; and estimating the metabolic flux according to sampled values of a plurality of batches of extracellular metabolite concentrations.

In an embodiment, the metabolic flux changes with time, and a metabolic flux change pattern between two adjacent time points is acquired to describe transient features of the metabolic flux, the transient features including a constant, a linear function, and a quadratic function.

In an embodiment, the sampled values of the plurality of batches of extracellular metabolite concentrations are selected according to production practice, sampling is performed once or sampling is performed a plurality of times within a calculation time interval, and then curve fitting is performed.

In an embodiment, during estimation of the metabolic flux, an optimal metabolic flux is calculated by using an optimization algorithm of calculation of a derivative, linear planning, and quadratic planning.

In an embodiment, the establishing a biological fermentation process monitoring model includes: combining the production process variable and the metabolic flux in the cells to form and extend a sample dataset; standardizing the sample dataset to generate a standard sample dataset; establishing the monitoring model through a principal component analysis; and determining a control limit for a monitored statistical indicator.

In an embodiment, during extension of the sample dataset, a complete metabolic flux or an intracellular and extracellular exchange metabolic flux or an internal metabolic flux is selected for the metabolic flux in the cells.

In an embodiment, the principal component analysis includes a general principal component analysis, a multi-stage principal component analysis, a kernel principal component analysis, and a support vector machine method.

In an embodiment, the online running status monitoring includes: acquiring an extracellular metabolite concentration of a current batch at a current moment and the production process variable; estimating the metabolic flux by using extracellular metabolite concentration; combining the production process variable and the metabolic flux to form a test dataset; standardizing the test dataset to generate a test standard dataset; pre-estimating and filling test data at a future moment; and calculating a statistical standard, and performing comparison with the statistical quantity control limit.

In an embodiment, a method for pre-estimating and filling test data at a future moment includes pre-estimating and filling future data by directly using average data of moments corresponding to previous several batches or by considering differences between actual data of a current moment and a previous moment and corresponding average data.

Compared with the prior art, the foregoing technical solution of the present invention has the following advantages:

In the present invention, in the method for monitoring a biomanufacturing process using a metabolic network of cells, based on data of a plurality of batches of production processes, a metabolic flux of cells is estimated by using a metabolic network of the cells and a concentration of an extracellular observable metabolite, and the metabolic flux is combined with a production process variable to construct and extend a sample dataset, to provide a method for online monitoring of running status of a process. The method uses detectable or observable information in a microbial manufacturing process, and fully exploits undetectable intracellular metabolic information in a growth and reproduction process of cells, so that running status evaluation and fault monitoring of a production and manufacturing process can be implemented more effectively. Anomalies can be discovered in time during process running, and corresponding control measures can be implemented, which significantly reduces potential safety hazards and improves comprehensive benefits of enterprises.

### BRIEF DESCRIPTION OF THE DRAWINGS

To make the content of the present invention clearer and more comprehensible, the present invention is further described in detail below according to specific embodiments of the present invention and the accompanying draws. Where:

FIG. 1 is a step chart of a method for monitoring a biomanufacturing process using a metabolic network of cells according to the present invention;

FIG. 2 is a schematic flowchart of a method for monitoring a biomanufacturing process using a metabolic network of cells according to the present invention;

FIG. 3 shows calculation results of concentrations of some observable metabolites of a penicillin fermentation process; and

FIG. 4 shows dynamic estimation of some metabolic fluxes of a penicillin fermentation process.

### DETAILED DESCRIPTION OF THE PREFERRED EMBODIMENTS

The present invention is further described below with reference to the accompanying drawings and specific embodiments, to enable a person skilled in the art to better understand and implement the present invention. However, the embodiments are not used to limit the present invention.

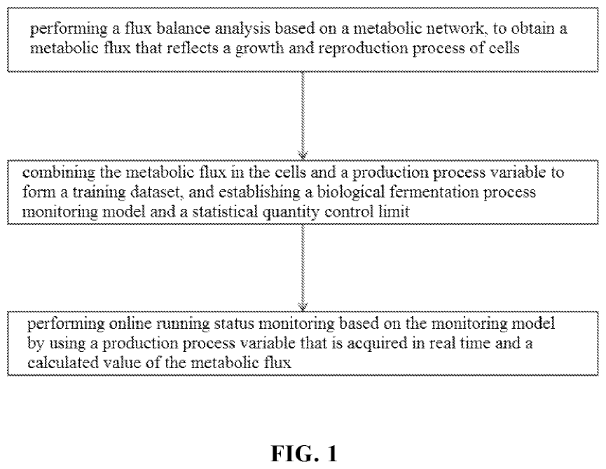

Refer to FIG. 1. A method for monitoring a biomanufacturing process using a metabolic network of cells in the present invention includes the following steps: performing a flux balance analysis based on a metabolic network, to obtain a metabolic flux that reflects a growth and reproduction process of cells; combining the metabolic flux in the cells and a production process variable to form a training dataset, and establishing a biological fermentation process monitoring model and a statistical quantity control limit; and performing online running status monitoring based on the monitoring model and by using a production process variable that is acquired in real time and a calculated value of the metabolic flux. Based on data of a plurality of batches of production processes, a metabolic flux of cells is estimated by using a metabolic network of the cells and a concentration of an extracellular observable metabolite, and the metabolic flux is combined with a production process variable to construct and extend a sample dataset, to provide a method for online monitoring of running status of a process. The method uses detectable or observable information in a microbial manufacturing process, and fully exploits undetectable intracellular metabolic information in a growth and reproduction process of cells, so that running status evaluation and fault monitoring of a production and manufacturing process can be implemented more effectively. Anomalies can be discovered in time during process running, and corresponding control measures can be implemented, which significantly reduces potential safety hazards and improves comprehensive benefits of enterprises.

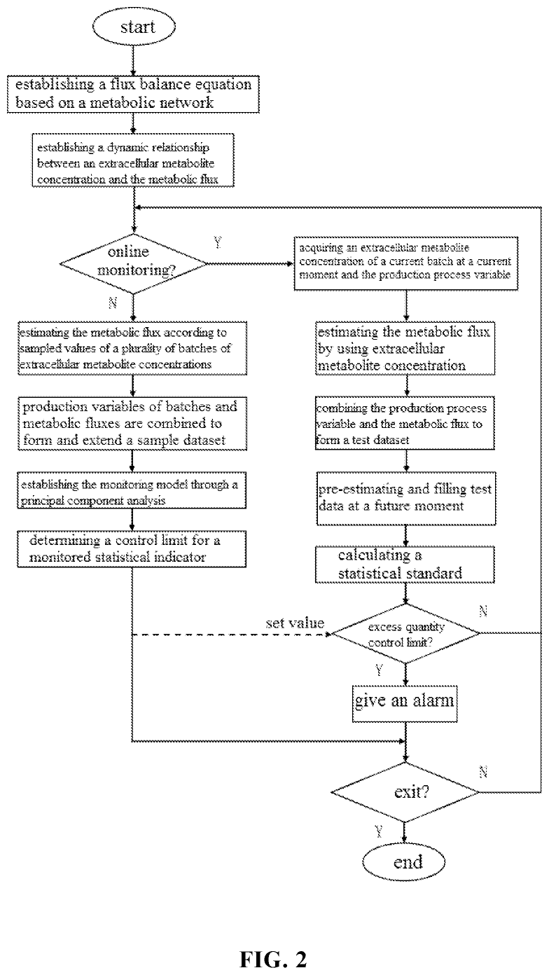

Refer to FIG. 2. A process of the flux balance analysis includes: establishing a flux balance equation; establishing a dynamic relationship between an extracellular metabolite concentration and the metabolic flux; and estimating the metabolic flux according to sampled values of a plurality of batches of extracellular metabolite concentrations. The establishing a biological fermentation process monitoring model includes: combining the production process variable and the metabolic flux in the cells to form and extend a sample dataset; standardizing the sample dataset to generate a standard sample dataset; establishing the monitoring model through a principal component analysis; and determining a control limit for a monitored statistical indicator. The online running status monitoring includes: acquiring an extracellular metabolite concentration of a current batch at a current moment and the production process variable; estimating the metabolic flux by using extracellular metabolite concentration; combining the production process variable and the metabolic flux to form a test dataset; standardizing the test dataset to generate a test standard dataset; pre-estimating and filling test data at a future moment; and calculating a statistical standard, and performing comparison with the statistical quantity control limit.

### Establish a Flux Balance Equation:

A net accumulation or consumption rate of an intracellular metabolite in a microbial fermentation process is far less than a rate of exchange reactions between intracellular and extracellular metabolites. For a metabolic network that has a total quantity of chemical reactions being F and that includes m intracellular metabolites and R extracellular observable metabolites, a flux balance equation of the metabolic network is as follows:

$${\dot{C}}_{R π} = {S}_{R} \cdot {V}_{t} , {S}_{m} \cdot {V}_{t} = 0 \qquad ( 1 )$$

C_{R,x}∈R^{R} denotes a concentration vector of the R observable extracellular metabolites at a moment t. S_{R}∈^{R×F} denotes a chemical measurement matrix of the extracellular observable metabolites. Vₜ∈R^{F} denotes a metabolic flux vector in the metabolic network, and includes an intracellular metabolic flux and an intracellular and extracellular exchange flux. Sₘ∈R^{m×F} denotes a chemical measurement matrix of the m intracellular metabolites.

The quantity F of chemical reactions in a microbial metabolic network is greater than a quantity m of metabolites in the cells, and has a degree of freedom of d=F−rank(Sₘ). A linear combination of a group of independent fluxes uₜ∈R^{d} is used to denote a metabolic flux, that is, Vₜ=K·uₜ. K∈R^{F×d} denotes a group of standard orthogonal bases of a null space Sₘ. uₜ is referred to as a free flux.

Describe a transient feature of a metabolic flux:

It is taken into consideration that the metabolic flux Vₜ changes with time. A calculation time sequence point to is introduced, D=1, . . . , Z, and is used for describing and analyzing a dynamic change of Vₜ. That is, an entire production process is divided into Z−1 time intervals. In each time interval, that is, between two adjacent time points t_{D} and t_{D+1}, it is assumed that the metabolic flux changes according to a specific function pattern, which may be a constant, a linear function, or a quadratic function. The metabolic flux Vₜ after the linear function is chosen is denoted as follows:

$${V}_{t} = {\sum }_{D = 1}^{Z} K \cdot {u}_{D} \cdot k ( t , {t}_{D} ) \qquad ( 2 )$$

u_{D} denotes a free flux of a D^{th} calculation moment. k(t,t_{D}) is a coefficient function of a linear change of the metabolic flux in time interval, and is selected according to Table 1.

TABLE 1

| | Coefficient function |
|---|---|
| Time interval | k(t, t_{D}) |
| D = 1 & t = t_{D} | 1 |
| D > 1 & t ≤ t_{D-1} | 0 |
| D > 1 & t_{D-1} < t ≤ t_{D} | $\frac{t - {t}_{D - 1}}{{t}_{D} - {t}_{D - 1}}$ |
| D < Z & t_{D} < t ≤ t_{D+1} | $1 - \frac{t - {t}_{D}}{{t}_{D + 1} - {t}_{D}}$ |
| D < Z & t_{D+1} < t | 0 |

Calculate an extracellular metabolite concentration based on a metabolic flux: Formula (2) is substituted into Formula (1), to obtain:

$${\dot{C}}_{R , t} = {\sum }_{D = 1}^{Z} {S}_{R} \cdot K \cdot {u}_{D} \cdot k ( t , {t}_{D} ) \qquad ( 3 )$$

Integration is performed on two sides of Formula (3) to obtain:

$${C}_{R , t} = {C}_{R 0} + {\sum }_{D = 1}^{Z} {S}_{R} \cdot K \cdot {u}_{D} \cdot γ ( t , {t}_{D} ) \qquad ( 4 )$$

The function is γ(t, t_{D})=∫₀^{t}k(t, t_{D})dt C_{R0}=[C_{1.0}, C_{2.0}, . . . , C_{R,0}]^{T} denotes a vector of initial concentrations of the R extracellular observable metabolites.

In a production process, an actual sampling time point is tₙ, n=1, . . . , N. According to Formula (4), a calculated value of a concentration of an r^{th} extracellular metabolite (r=1, . . . R) at an actual sampling moment t′ₙ is:

$${C}_{r , n} = {C}_{r , 0} + {\sum }_{D = 1}^{Z} {S}_{R} \cdot K \cdot {u}_{D} \cdot γ ( {t}_{n}^{′} , {t}_{D} ) \qquad ( 5 )$$

C_{r,0} denotes an initial concentration value of the r^{th} extracellular metabolite.

A sampling point may be selected according to production practice. A simple operation is to perform sampling once in each calculation time interval of the metabolic flux Vₜ, that is, Z=N−1. Alternatively, sampling may be performed a plurality of times in a calculation time interval. Generally, the first calculation time point and the last calculation time point of the metabolic flux Vₜ should respectively correspond to an initial moment and a final moment of actual sampling.

Estimate of a metabolic flux at each sampling moment:

At the sampling time point tₙ, an actual sampled value of each extracellular metabolite is C′_{r,n}. A deviation of a calculated value of a concentration from the sampled value is:

$$Φ = {\sum }_{r = 1}^{R} {\sum }_{n = 1}^{N} [ {σ}_{r , n}^{- 2} \cdot {( {C}_{r , n} - {C}_{r , n}^{′} )}^{2} ] \qquad ( 6 )$$

σ_{r,n}² is a concentration error item variance of an extracellular metabolite r at a sampling moment n.

U=[u₁^{T}, u₂^{T}, . . . , u_{z}^{T}]^{T} denotes a d-dimensional free flux at calculation moment. An optimization algorithm is used to search for U that meets Formula (5), so that the deviation Φ is minimal. That is,

$${min}_{U} Φ$$

is calculated to obtain the optimal free flux U, that is, the free flux u_{D} at each moment t_{D}, and the metabolic flux Vₜ is then estimated by using Formula (2).

A derivative of U, that is,

$$\frac{\partial Φ}{\partial U} ,$$

is directly calculated from Formula (6) by using a optimization algorithm, to obtain an optimization variable U=H⁻¹·J. A matrix H and a matrix J are given by using the following Formulas:

$$H = {\sum }_{r = 1}^{R} {\sum }_{n = 1}^{N} [ {( \frac{\partial {C}_{r , n}}{\partial P} )}^{T} \cdot {W}_{r} \cdot ( \frac{\partial {C}_{r , n}}{\partial P} ) ] \qquad ( 7 )$$

$$J = {\sum }_{r = 1}^{R} {\sum }_{n = 1}^{N} [ {( \frac{\partial {C}_{r , n}}{\partial P} )}^{T} \cdot {W}_{r} \cdot {C}_{r , n}^{′} ] \qquad ( 8 )$$

Wᵣ in the formulas is a diagonal matrix formed by σ_{r,n}⁻².

In engineering practice, an initial concentration Cao and a metabolic flux calculation moment t_{D} may be added as optimization variables, and

$${min}_{U , {C}_{0} , {t}_{D} ,} Φ$$

is calculated by using the optimization algorithm, to obtain the optimal free flux U, initial concentration C₀ and moment t_{D}.

Establish a running status monitoring model and an indicator:

### Extend a Sample Dataset

In existing I normal batches of data in production, a data matrix of an i^{th} batch of production variables (including a temperature, a pH, a ventilation rate, a stirring power, a substrate feed rate, and the like) is Xᵢ∈R^{N×O}, i=1, . . . l, N is a quantity of times of sampling point, and O is a quantity of production variables. According to the estimated metabolic flux Vₜ, all or some metabolic fluxes are selected. If data of L intracellular and extracellular exchange metabolic fluxes in an i^{th} batch is selected to form a set Yᵢ∈R^{N×L}. Production variable data of the batch and the intracellular and extracellular exchange metabolic fluxes are combined to form and extend a sample dataset Gᵢ=[Xᵢ, Yᵢ]^{T} ∈R^{N×H}, wherein H=O+L. A sample dataset of all I batches of fermentation processes extended is G̅=[G₁, G₂, . . . , G_{I}]∈R^{I×N×H}, and is expanded according to a batch direction to obtain G∈R^{I×N·H}.

### Standardize a Dataset

An extended sample dataset is standardized and converted into a standard training dataset with an average of 0 and a variance of 1, which approximately conforms to a multi-dimensional normal distribution:

$${G}_{train} = \frac{G - mean ( G )}{std ( G )} \qquad ( 9 )$$

mean(G) is an average of a same variable in batches at a same moment in G, that is, an average running trajectory in a plurality of batches of normal operations. std(G) is a standard difference of a same variable in batches at a same moment in G.

Calculate a monitoring model and a control limit

A covariance matrix

$$\sum = \frac{1}{I - 1} {G}_{train}^{T} {G}_{train}$$

of a standard dataset Gₜᵣₐᵢₙ is constructed. A monitoring model is established by using a principal component analysis. A feature value of the monitoring model and a feature vector of the monitoring model are first calculated. In descending order of accumulated variance contribution ratios, feature vectors q₁, . . . , q_{A} corresponding to A principal component feature values are selected to form a payload matrix Q=┌q₁, . . . , q_{A}┐, to calculate a score vector and residual vector of each batch of Gₜᵣₐᵢₙ. Score vectors and residual vectors of all I batches respectively form a score matrix and a residual matrix, and a common T² control limit and a common SPE control limit are accordingly calculated.

Establish the monitoring model may also be used to perform a method such as a multi-stage multi-direction principal component analysis, a multi-direction kernel principal component analysis, and a multi-class support vector machine.

Online real-time running status monitoring:

### Process Data at a Current Moment

Production variable data X_{test,n}∈R^{n×O} of a production process at a current moment n is acquired. By using an estimated metabolic flux Y_{test,n}∈R^{n×L} a production variable and the metabolic flux are combined to obtain a test dataset G_{test,n}∈R^{n×H}. An average value and a standard difference at a corresponding moment in a training dataset are used to standardize G_{test,n}∈R^{n×H}.

### Estimate Data at a Future Moment

Data from an (n+1)^{th} moment after a current moment to an N^{th} moment at which a batch ends is filled by using a pre-estimation method. A data value at an n^{th} moment in standardized G_{test,n} to perform filling, to obtain G′_{test,n}∈R^{N×H}, and then expansion is performed in a time direction to obtain G″_{test,n}∈R^{1×N·H}. Alternatively, future data may be directly pre-estimated and filled by using average data at moments corresponding to the first several batches or considering differences between actual data at a current moment and a previous moment and corresponding average data.

Calculate a T² statistical quantity and an SPE statistical quantity at a current moment

A score vector t_{test,n} and a residual vector e_{test,n} at a current moment n are calculated according to a payload matrix Q of a standard training dataset Gₜᵣₐᵢₙ:

$${t}_{test , n} = {G}_{test , n}^{″} \cdot Q \qquad ( 10 )$$

$${G}_{new , n} = {t}_{test , n} \cdot {Q}^{T}$$

$${e}_{test , n} = {G}_{test , n}^{″} - {G}_{new , n}$$

The T² statistical quantity and the SPE statistical quantity at the moment n are calculated according to the following formula:

$${T}^{2} = {t}_{test , n} \cdot {Ψ}^{- 1} \cdot {t}_{test , n}^{T} \qquad ( 11 )$$

$${SPE}_{n} = {e}_{test , n} \cdot {e}_{test , n}^{T}$$

Ψ∈R^{A×A} is a diagonal matrix formed by elements in an i^{th} batch of score vectors of Gₜᵣₐᵢₙ.

### Monitor a Running Status of a Process

If T² and SPEₙ are less than statistical quantity control limits, running of the process is normal; or otherwise it is pre-warned that the process has an anomaly or a fault.

To verify the effect of performing the method for monitoring a biomanufacturing process using a metabolic network of cells in this embodiment, the method is applied to monitoring of running of a penicillin production process:

A penicillin fermentation metabolic network includes a total of 66 metabolic fluxes (five intracellular and extracellular exchange fluxes) and 49 intracellular metabolites. Five extracellular metabolites, namely, glucose, penicillin, bacterial biomass, oxygen, and carbon dioxide, are detectable. A total of 11 production process variables and intracellular and extracellular exchange metabolic fluxes such as reaction temperature, a pH value, a ventilation rate, a stirring power, a substrate feed rate, and a cooling water feed rate are considered. 30 batches are selected. Each batch has 800 sampling points, to form and extend a sample dataset.

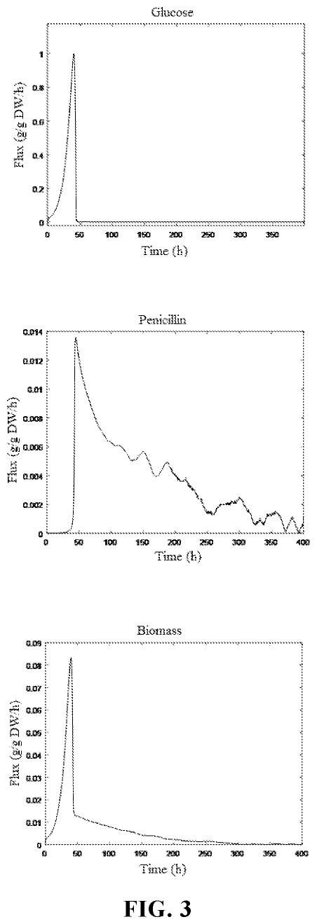

Calculation of an extracellular metabolite concentration of a metabolic flux and estimation of a metabolic flux at each sampling moment are completed according to the method of the present invention. FIG. 3 provides an estimated value of an intracellular and extracellular exchange metabolic flux, so that an extended sample dataset includes detectable production process variable information in a biological fermentation process and also includes unobservable metabolic information inside cells, so that features of a production process are reflected more comprehensively.

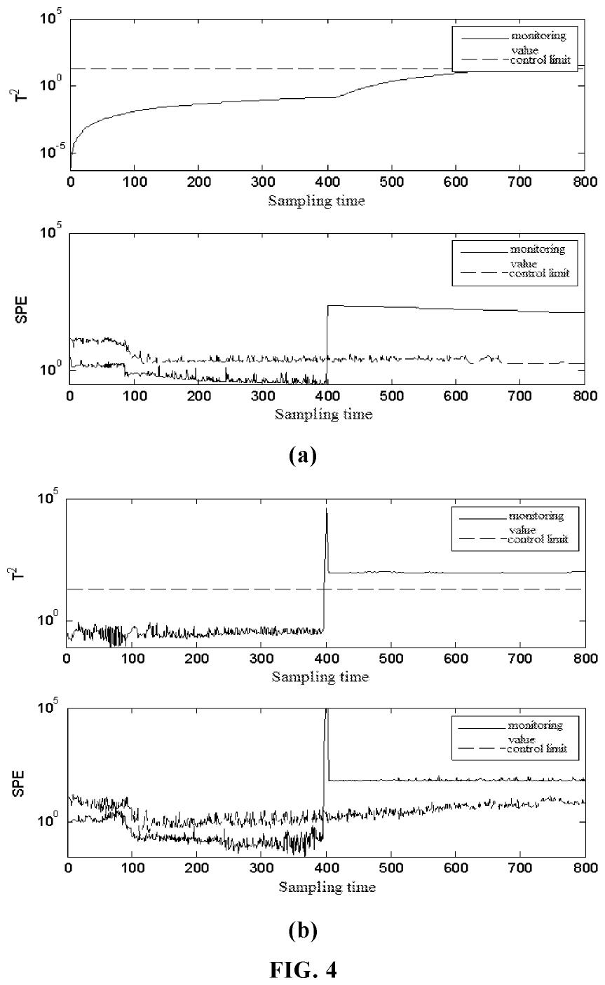

For typical faults caused by a ventilation, stirring, and substrate feed in a penicillin fermentation process, FIG. 4 provides results of online monitoring according to the present invention:

Only production process variables are used in process monitoring denoted by (a). Metabolic network information is combined in (b). As can be learned from the monitoring diagram of T², when a fault occurs, the method provided in the present invention can accurately monitor a fault in time, and monitoring based on production variables has a specific time lag.

Persons skilled in the art should understand that the embodiments of this application may be provided as a method, a system, or a computer program product. Therefore, this application may use a form of a hardware-only embodiment, a software-only embodiment, or an embodiment with a combination of software and hardware. In addition, this application may use a form of a computer program product implemented on one or more computer-usable storage media (including, but not limited to, a disk memory, a CD-ROM, an optical memory, and the like) that include computer-usable program code.

The present application is described with reference to the flowcharts and/or block diagrams of the method, the device (system), and the computer program product according to the embodiments of the present application. It should be understood that computer program instructions may be used to implement each process and/or each block in the flowcharts and/or the block diagrams and a combination of a process and/or a block in the flowcharts and/or the block diagrams. The computer program instructions may be provided for a general-purpose computer, a dedicated computer, an embedded processor, or a processor of another programmable data processing device to generate a machine, so that the instructions executed by the computer or the processor of the another programmable data processing device generate an apparatus for implementing a specific function in one or more procedures in the flowcharts and/or in one or more blocks in the block diagrams.

These computer program instructions may be stored in a computer-readable memory that can instruct the computer or any other programmable data processing device to work in a specific manner, so that the instructions stored in the computer-readable memory generate an artifact that includes instructions apparatus. The instruction apparatus implements a specific function in one or more processes in the flowcharts and/or in one or more blocks in the block diagrams.

The computer program instructions may alternatively be loaded onto a computer or another programmable data processing device, so that a series of operations and steps are performed on the computer or the another programmable device, to generate computer-implemented processing. Therefore, the instructions executed on the computer or the another programmable device provide steps for implementing a specific function in one or more procedures in the flowcharts and/or in one or more blocks in the block diagrams.

Obviously, the foregoing embodiments are merely examples for clear description, rather than a limitation to implementations. For a person of ordinary skill in the art, other changes or variations in different forms may also be made based on the foregoing description. All implementations cannot and do not need to be exhaustively listed herein. Obvious changes or variations that are derived there from still fall within the protection scope of the invention of the present invention.

What is claimed is:

1. A method for monitoring a biomanufacturing process using a metabolic network of cells, comprising steps of: performing a flux balance analysis based on a metabolic network, to obtain a metabolic flux that reflects a growth and reproduction process of cells; combining the metabolic flux in the cells and a production process variable to form a training dataset, and establishing a biological fermentation process monitoring model and a statistical quantity control limit; and performing online running status monitoring based on the monitoring model by using a production process variable that is acquired in real time and a calculated value of the metabolic flux.

2. The method for monitoring a biomanufacturing process using a metabolic network of cells according to claim 1, wherein a process of the flux balance analysis comprises: establishing a flux balance equation; establishing a dynamic relationship between an extracellular metabolite concentration and the metabolic flux; and estimating the metabolic flux according to sampled values of a plurality of batches of extracellular metabolite concentrations.

3. The method for monitoring a biomanufacturing process using a metabolic network of cells according to claim 2, wherein the metabolic flux changes with time, and a metabolic flux change pattern between two adjacent time points is acquired to describe transient features of the metabolic flux, the transient features comprising a constant, a linear function, and a quadratic function.

4. The method for monitoring a biomanufacturing process using a metabolic network of cells according to claim 2, wherein the sampled values of the plurality of batches of extracellular metabolite concentrations are selected according to production practice, sampling is performed once or sampling is performed a plurality of times within a calculated time interval, and then curve fitting is performed.

5. The method for monitoring a biomanufacturing process using a metabolic network of cells according to claim 2, wherein during estimation of the metabolic flux, an optimal metabolic flux is calculated by using an optimization algorithm of calculation of a derivative, linear planning, and quadratic planning.

6. The method for monitoring a biomanufacturing process using a metabolic network of cells according to claim 1, wherein the establishing a biological fermentation process monitoring model comprises: combining the production process variable and the metabolic flux in the cells to form and extend a sample dataset; standardizing the sample dataset to generate a standard sample dataset; establishing the monitoring model through a principal component analysis; and determining a control limit for a monitored statistical indicator.

7. The method for monitoring a biomanufacturing process using a metabolic network of cells according to claim 6, wherein during extension of the sample dataset, a complete metabolic flux or an intracellular and extracellular exchange metabolic flux or an internal metabolic flux is selected for the metabolic flux in the cells.

8. The method for monitoring a biomanufacturing process using a metabolic network of cells according to claim 6, wherein the principal component analysis comprises a general principal component analysis, a multi-stage principal component analysis, a kernel principal component analysis, and a support vector machine method.

9. The method for monitoring a biomanufacturing process using a metabolic network of cells according to claim 1, wherein the online running status monitoring comprises: acquiring an extracellular metabolite concentration of a current batch at a current moment and the production process variable; estimating the metabolic flux by using the extracellular metabolite concentration; combining the production process variable and the metabolic flux to form a test dataset; standardizing the test dataset to generate a test standard dataset; pre-estimating and filling test data at a future moment; and calculating a statistical standard, and performing comparison with the statistical quantity control limit.

10. The method for monitoring a biomanufacturing process using a metabolic network of cells according to claim 9, wherein a method for pre-estimating and filling test data at a future moment comprises pre-estimating and filling future data by directly using average data of moments corresponding to previous several batches or by considering differences between actual data of a current moment and a previous moment and corresponding average data.
